SEQUENCE LISTING (8 sequences)

SEQ ID NO: 1 - DNA, 1956 bp, Zea mays
  source 1..1956: mol_type = other DNA
aggctatgtccagtaacgctatctctatcatctctccaatcatatcatttatcctattttttattttaaatttatcatctatatatttttacacgatcatataaacattataatgaaacgtatagaaaaaagaattttagtcactatatataaaaacacagatatgaagatattaacctcatgtactagcaactccaaaaggctctatatattcttcctaatctatataaatatattttttaaaatatatatatatatatctttcaataacttatctaaatgactatccataaatatagacatcttatattttaggtttctcgctattcaaatatagaaaacatgaatgattcttagaatacaaacaagatatagactgttagagattaaaaagatgtagaaaatggtttttatacaaataattatttaaataatgattaaatgataacttaaagagtgaaatttagaatgactcaagacgttctaaatttaggttctctcctctctgtatcgacgaaattttggcttttgttctttctctctaccttggccgcgttcgcacgtgctcgctgccctagatgatttttttttgtttgtgacatgattaagggcttgaggattagtatggattggaaattattttggtttactaaaagtatcttaagagactctttatttttaactctcaatttaactctttatttgtttatttctctataaatattaaaatttatatataacatctcattacaacagactctctaacatacaagttaacttggccagcgagagttgctagctaaatttggttagtgagaaagttaatttatagaaccagataacttagaaagttaaatgactagtctgttgaagagctattatgttattaagtggctaaaatttagtttgacaaactatttaactagtttattggagatactctaaagatttaaagtcatccgatctctaacgatcccatgaattaagattctaaagggtctaggtgtcgataccatccgtattcaggcgtccgttgaaaatagtcttacagctagggttggatatcgagtaaatttgactcgcctcgtccgagtttaaactggctcgttaaactaacgaaccacatagtcagcttgactcgactcatttactagctcgtttaggttgcgatcctaagtaaaaaaacaatatgtatataaaataatccatttctagttaattttaactaatctaataatataaaaattatgcttatacccatagtttcacataccacatcaatgtaacattaaactgatacaattcatcacttgtcaatttacaatttataattcacatatatattcgtcagtttaataatagttatattcgtatagaccaatttaacatgtagacatagttcatcaataattagtttaactcatgagtaataaacactacacataaatataacatattcatcaattattactatatcaattattactatatatatatatagttttgttttgctaaaatataatacctccttgtttagctcacaaaatagctccttacaaaccaagcttgaatatcagtttaacttataaaaaaattcaacgagtcagagttaaattgagctcacgagcatttcgttctgtctacttctaccatcaagcaccaaaaccagcggagcgggccggacgagggacgacaagaaaccatttctggtacggctcgcgacacgttacagcctagcagggacggccgccatgcacgcgcccacgtcgcgtccgctgacgcgccacgtcccgcgccaccgccgagccgctttatatatctgtactacaccgcagccggcgcagcggaatacacgccagaaactaatccagatagctagcctttgatttctcacttcaatctgacgaagtgcacacctaggcaacaaactcgaacgcgcgcggtagtggagccgaa

SEQ ID NO: 2 - DNA, 1860 bp, Zea mays
  source 1..1860: mol_type = other DNA
aaggcggactatggcatttggggtcagttgatgcatgaatacctggtgcttttgcaatatcattccaatcaatttatacatgggcagctgaagtccagccttgaaaaagctcctgtagacgacaactttgtctttcttgggcttcagagttgtctcttctccagggagtcatacgttgaccttgttgctaaagtaaccaagctttttcatcgtttgcaaatctttctccttcagtgtgtaccggcccatctcaatgtgacttggtttcgatggcctatccttcggcaactcctccacaggctatgctaaggacgaagccttcatggcttcggaattcagcaaagtatcctaaagtgcctccgaagaagtcagcgtggttagtctagcagctgagtggatcctagccatctagacaatttctacccgaaggtgttttccctaaactagtcatcgaggcagtggtggagaaaaagcttcagagtcgcagaaacttttggcagaaagtgggtgagggcagtaaatgtgactcactcacacccgctccgctgcttttatactgagaattgggtgcgaaagggcgcgagcagcgaaaaaaaatctgcacacgtggacgttggccttcccaccttctccctgtctggtggaacacgagcttggcttcatttgtaccaaagtattcatttaaccttttccataacgagctttgggaaggtgttcttcggacatttagctcaaggactactagatatatttttgtgtttcgagctcgttatgaaaaaatgaatttatcccgcaagaggctactgttgggggcctagttgcccgaagatcctcaaaatagctaaccttcgacataaaaagatgtatttcaggcacataaggaagggtggcgaagatgaagtctgaagtcaccagcttcggcgtgccaaaggcttcgtgtgcggaagcgacgaaggagaaaaggttgaagtcttgtgcagcaagaaagagagaaaaaactttgatgccccatgatatttgtaaatcataattgttaactttgtgggcatacttataatttcatacaaagctgtacctctcccctataaatagatgaacagtgtcatgcataaatgctccttttggagggcttcaagcttcatctctcttacccttcgaagtacattctagttgtttttttgtgtaaaaccgaatgtatgcttgtacatttaattcataagaagggtgaaataagagaatgccaaggcatacaaggtgactagcttgcttcgtccttaatcacacttttatacatccttacttcgcctgtgtccttcgtttattccttctcccttagacgaagaaagtttcaatcctctaacttgtgttgccttgtttttgatatcatataacaatttaaaacaagtgaccaacaattatatattccatcctatccaaagtataattcgttacactaattacacattctttaagtaatatataaatttagtttgtatgtatgtctatattcattatcatttaaatgagtgtgtacagaaaaaataagctagaaagaactatattttaggaggcaggaagtatatttttttataagaactgaatgaatagtttaccgataaacaccgcgtcataagcagagcgcgggcgcgtgccacagccactgcccgcagcttcgacatgtgcatgtggtggacgcagtccggtgggcggtggctgcgcatcaaccaatcaccggccgccttcctcgccacacggggcagagatatagttaggctaggccccgggcggctcatccgcaagccagtggacgccagtgagtgaacaccagccgtcttggcccgctccttccctgctgcggca

SEQ ID NO: 3 - DNA, 1925 bp, Zea mays
  source 1..1925: mol_type = other DNA
aagtcggttcccttttctgcgaacatgccgaagctttcctgcttcacagcttcggcgctgtgtcaacctttgtagcttttccctttctgatacgagtccgaagatacctgctcacacattttaagccgcttctcccttgtagaccttcggcgacgaagcatagacccaacagatacctgttcacacattatactccagaaatactgttaaatcctgtttttgaggaccttcggatgccgaaggtccccaacaatcggtattctaaaatatcagaaaataagtttgttatagaaaatacatcgactatgatcgaaatctaaaatatgattgataaaaatcgtaacatgtaccctaaattgatttaaaaagtcacataagttagtaataatataaatattcacaaatttatgaaggattaaagcatatctcataaaagcaataaaacaatatctcacaaaatacaagtggcaaacattatacaaacatacacatagtcagaaagtcacaactcaggaccttaaaaaatgaaactatccgattgaaaatacattgataacaattgaacactagaaaataatatcacaaatcaaactatggagcatataactagccatataactcttataatacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtcacgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgaa

SEQ ID NO: 4 - DNA, 1840 bp, Zea mays
  source 1..1840: mol_type = other DNA
gtgctatggttgtggacctgccttctgctacgatgaaggacactgataaaattagaagagggttcctttgatatggttgtaaaaatgccatgggtggacattgtgcagtggcatggggaagagtgtgcagactcacgagcttggtgggctgggtattttcagcatcactgagctgagttgggctcttaaaatccgatgggcttggctgcaaaaaactgagccatgtcagccctggtcatccctgccccttcatatcctgaggaaaatccgagagtttttacaaatggccatgtattctgagattggaaatggagcctctaccctcttctggagtgatcgatgactgggtgggcagcggatagtggatattgctcctcgtcttttagggaccattcctaagaagctgattaataaaaggacagttcagaaggcgatcggagataatttatggattaatgatattcctaggagtttatctgttggagcacttgcagactttctgcgcctttgggatttggtggcccaagttgatctttgccctgataaataggacaagcatatttttcggttggctgctaatggcaaatatccggctaaagctgcttatgaggggctcttcttgggtttagtggagttcgaaccttatgagaggatttggaaaacttgggctccgcccaaatgccaattcttcttgtggttggtggctcataagaaatgttggacgacagatatacttgaaaagcgaggtttgaatcatctgaaaagatgtcctctttgtgatcagaaaagggagacaatagaccatctgcttgtgacttgtgtcttttctagagaatgttggttcctccttcttaggcaattcagactccatggtccgcaaccatcgaatattaatatcatggagtggtggcatcaagcaaataaagctatttaggggtccttcagagatggcctaaactctctcattatgctaggtgcttgggtcttatggaatcaccaaaacagatgcatctttgatagtctttcccctaatattgctaatttcttaattcaaattggggaccatcactgtctgtgggagactgctggggccaaggggctgtcctcccttgctgccttcctctcagttgtggactaaagctttatagatgagtcagtttatgttcgttacgtttagacagccgtaaggcgtcaattgtaccctggtctattttggaccttttttctttctattttttatataatgatatgtaactctcctgcgtgttcgagaaaaaaaagataataatgagattaagggagaagatcggcatctcaggaggagatggtgagttgcctgaccaaggtcatgaagtggacgtgggcacgtggcaaatccactccaggaacggaccaaaaataggctaccactttcagggcgcgacgtggacgtattgaagatctggtggtcaccgcgaaatgtacacatcagaacgaggcatgaaactggccttggccgtggacgcgtgaagcgcgccatgcgcgtccgacgtggacgtaatgagctactacttcagagccacatgtgtccacgcttcatcagaaagaaaacacctgcacacgtcaactggagctgccatcagtttagtttacatgctgcatcgtctgaaggccagatcagtgatgagcgaaacattctatatataccctcagtccctgttccagttctcgtcaagctagcagcacgagttgtcgaacacttgcctgctcttgagctcaagctatcatcagctgcgtcttgtgcacagcaacagcttcccaaccgcgcgcaactgtaccagcgaaactaccaactgccata

SEQ ID NO: 5 - DNA, 999 bp, Zea mays
  source 1..999: mol_type = other DNA
cgcgaagtctgactggccagcccaaactcagcctttgtagtagagagagccgaccttggcggcctgcacgacatttttgaactttttttatcggtttggacttcattactttatattctttgtcaaccaaatactcccttcctcttaaattataattcgtttgattttttttatgtaaagttttatgtatagtccacctagttaactttaaaagttggttagtctagattttatggaggccgactacaatagcaaatctaacaataaaaaagttagctagatcagatttcaagaacagtctgaccgattttgaagatttgattcaggttaattctacgattttgaagatccgacctaacctaattttacgttcagactaaagacatgtttgtttaagattatgatatgtctagttatataatttaacttattttaaactaacacttaatttaaaataagttagattatatgatcagaatattatatttatataattccaaacaaataactctaatatcgattctggtttatattgtataatttttatgttgaatattacggcgcgccaacaaattgcgatcgttatgtaatcatgtatacgaccgactgtactattcataaaatataattcaaaataggagatgctcgagatagcctggcaagggaggaggtatcggaggcggcgtcgcctgccgcgagcgcaccatgcgtcaacgctcaggacctctcgcaggcgcgctgttggatcgtgagcaagcacgcgtggcaaatggcatgcattcctctcgtgcaagtgccgagctgagggtcccgacgcggggggcaggacgtggcagagcgccgcccaggcggacgccaagtgccgagctaccagggcaccttctttattccgtccttgctcagtcacacctcgctctcgctcactctcgccgtccgcacagccgctcatcgtcacccactgcctgccctctccctgcgccggccggtgccccgacgcgc

SEQ ID NO: 6 - DNA, 999 bp, Zea mays
  source 1..999: mol_type = other DNA
cggcaaagaagtctttgccgatgtactgtttgccgagccttctttgccgagtgttacactcggcaaagcctttgccgagtgtttttaaggcttcgccgagtgcttgcggcactcggcgaagaggttgattccggtagtgatgctgacacacacacactactgcactattatattataaataaataaaaagatattgcattcccatcagacgagactcttatatatttgcgcacctattgttgctacaaattaaggttagcaagaatagaatccatgcatatcaggcatgcaagaaaattacatgaatagttaggataaatttaatattttaaaatagatatatatgaaatttaatgttaatattttcttacgtgttatcaagcacattgttatcgacaagaataattttttgcataactgtttttacattatgtgctccaaaaaattaaatgaaaatatccgtatctggtttttatatctatcatttaagaagatatacgttagatttgaagtttatattttatgaatcttaacaagctcaatactaaaaacaagaatatgaatttgcacatatcttatttaattattcacggtcaaaggaaaagagaacaaaataacaggcacgcggatgagtatttgttttcatccctaaccaagaggatggattagttagagaaatatttatggacgtgtaaggaaagaaaaaagattaacaacctaacatgtgtaagtaaaggaaatatattcatatagaagaaaaggaaaattcaatcagctgtggaagaattaattagaaaccaggattaatgaaactcgaggaacgtcaatataatgatgtccttctgtaaatttctgtgctatggaaggtgcataggccggctataaatagatgccatgacaccatgcaccacaccccgcatctcctagccccagctagatctttggacacatatcgaaacagctctagctagcgcgctcgtcgtctaacctggcttccgg

SEQ ID NO: 7 - DNA, 400 bp, Zea mays
  source 1..400: mol_type = other DNA
cacattctttaagtaatatataaatttagtttgtatgtatgtctatattcattatcatttaaatgagtgtgtacagaaaaaataagctagaaagaactatattttaggaggcaggaagtatatttttttataagaactgaatgaatagtttaccgataaacaccgcgtcataagcagagcgcgggcgcgtgccacagccactgcccgcagcttcgacatgtgcatgtggtggacgcagtccggtgggcggtggctgcgcatcaaccaatcaccggccgccttcctcgccacacggggcagagatatagttaggctaggccccgggcggctcatccgcaagccagtggacgccagtgagtgaacaccagccgtcttggcccgctccttccctgctgcggcc

SEQ ID NO: 8 - DNA, 499 bp, Zea mays
  source 1..499: mol_type = other DNA
taaaaaaaaggtgaagaagggaggaagaggaaatcagaagcaaaaaatgggcaactttaggcccattatctcgatggtcacgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcga

(19) United States
(12) Patent Application Publication
(10) Pub. No.: US 2026/0152755 A1
(43) Pub. Date: Jun. 4, 2026

(54) ISOLATION AND CHARACTERIZATION OF NOVEL TISSUE-SPECIFIC PROMOTERS IN MAIZE

(71) Applicant: Wisconsin Alumni Research Foundation, Madison, WI (US)

(72) Inventors: Heidi Kaeppler, Oregon, WI (US); Nathaniel Schleif, Madison, WI (US)

(21) Appl. No.: 19/532,314

(22) Filed: Feb. 6, 2026

Related U.S. Application Data
(62) Division of application No. 18/473,015, filed on Sep. 22, 2023.
(60) Provisional application No. 63/408,946, filed on Sep. 22, 2022.

(51) Int. Cl. C12N 15/82

(52) U.S. Cl. CPC C12N 15/8223; C12N 15/8205

(57) ABSTRACT

The present invention provides constructs comprising a transgene operably linked to a novel tissue-specific maize promoter selected from ris2, Ihcb10, nas2, aa2m, and expb14. Methods of using the constructs to drive tissue-specific transgene expression in a maize plant are provided. Maize plants comprising the constructs or made by the methods are also provided.

### CROSS-REFERENCE TO RELATED APPLICATIONS

This application is a divisional application of U.S. application Ser. No. 18/473,015, filed Sep. 22, 2023, which claims priority to U.S. Provisional Application No. 63/408,946 filed on Sep. 22, 2022, the contents of each of which are incorporated by reference in their entireties.

### STATEMENT REGARDING FEDERALLY SPONSORED RESEARCH

This invention was made with government support under 1917138 awarded by the National Science Foundation. The government has certain rights in the invention.

### SEQUENCE LISTING

This application includes a sequence listing in XML format titled “2023-09-21_960296.04434_WIPO_Sequence_Listing_XML.xml”, which is 18,434 bytes in size and was created on Sep. 21, 2023. The sequence listing is electronically submitted with this application via Patent Center and is incorporated herein by reference in its entirety.

### BACKGROUND

Maize is a vital global crop with 197 M ha of land dedicated to its growth, second only to wheat. As dry weight, maize production totals 1,137 million tons, 50% higher than either wheat or rice. Much of this production goes into the feeding of cattle and creation of a range of products from 3D printing filament to ethanol. Maize production is projected to continue to grow as it has for decades. Challenging this growth, though, is continued ecological degradation and anthropogenic climate change, both of which necessitate the continued genetic improvement of maize and development of biotechnology tools to support it.

Transgenic plants were first introduced to the market in 1994 with the “Flavr Savr” tomato and have since grown year over year, with 439 transgenic events now commercialized. Among these, 152 are maize events, with traits ranging from enhanced ethanol production, herbicide resistance, and insect resistance (ISAAA). Most of these have relied on overexpression of the transgenes, turning on expression at high levels in all tissues. This approach is sufficient with single events for certain traits but is complicated when multiple events are combined (stacked) into a single genotype, which is commonly done in commercial lines from industry. Stacked transgenes may interfere with one another, inducing silencing in cases where a promoter is duplicated. Transgenic approaches to crop improvement are increasingly more ambitious, such as introduction nitrogen fixation and C4 photosynthesis pathways into new crops. In tandem with and fueling these direct applications is the ever-expanding field of functional genomics research. Next generation sequencing has brought to the forefront many causative genes, especially through GWAS studies.

Modification of the maize genome has been used to confer agriculturally valuable traits to maize, such as pesticide or drought resistance. To express and study genes underlying these traits optimally, gene regulatory elements, like promoters, must often be used in conjunction with a transgene to control its temporal and spatial expression. There is a need for diverse promoters which are well characterized to drive gene expression. Currently, there are very few characterized, tissue-specific promoters that are available for use in maize, which limits genetic research and enhancement efforts of this crop.

While promoters that drive whole-plant (i.e., constitutive) expression in maize are known, they are not appropriate for use in cases where activity of the transgene outside of the target tissue negatively impacts growth. The phenotype resulting from expression of a transgene is often only needed in one specific tissue of the plant. For example, pesticide resistance is most important in the leaves and stalks of the plant, while drought resistance is most important in the roots of the plant, where water uptake occurs. Thus, the use of tissue-specific promoters is beneficial because it allows researchers to express a transgene in one tissue of the plant while leaving the rest of the plant unaffected. Thus, there is a need in the art for a toolbox of tissue-specific maize promoters.

### SUMMARY

Six tissue-specific promoters were identified and characterized in maize and demonstrated to control leaf-, embryo-, and root-specific gene expression. Promoters are a foundational biotechnology tool that enables scientists to both dissect gene function and bestow novel properties through genetic engineering. While many promoters have been developed and tested in model species such as Arabidopsis thaliana, crops like maize (Zea mays L.) still lack well-characterized options. Several promoters were identified from genes showing tissue-specific expression via analysis of RNA-seq data collected in maize across the entire development of the plant. Subsequently the promoter regions were cloned and transformed into inbred maize line, LH244, where qualitative and quantitative assessments demonstrated both tissue-specific expression, and instances of off-target expression. Finally, some promoters were further developed into shortened, “minimal promoter” forms and one promoter, Ihcb10, was demonstrated to function as a mesophyll-specifying cis-regulatory region when combined with the CaMV35S core promoter sequence.

In a first aspect, the present invention provides constructs comprising a promoter operably linked to a transgene. The promoter included in the constructs is the promoter of a maize gene selected from the group consisting of ris2, Ihcb 10, php20719a (clo2b), nas2, aa2m, expb14, and fragments of these promoters as well as the combination of elements of the Ihcb10 or php20719a promoter region in combination with the CaMV35S promoter).

In a second aspect, the present invention provides methods for driving expression of a transgene in a maize plant. The expression may be tissue specific. The methods comprise introducing a construct described herein into the maize plant.

In a third aspect, the present invention provides transgenic maize plants comprising a construct described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of the vectors used for transformation and testing of the candidate promoters. The two vectors differ only in the promoter used to drive expression of the visual marker gene tdTomato.

FIG. 2 shows expression levels of RNA transcripts of candidate tissue-specific genes in B73 and LH244 as measured by RNA-seq. The top panel features RNA transcript expression of the gene Ihcb 10 and the bottom panel features fl3. Each shows expression in transcripts per million across V1 stem and shoot, V1 pooled leaves, V7 crown roots, R1 12^{th} leaf, VT anthers, 12 days after pollination (DAP) whole seed, and 24 DAP embryo. Measurements are a mean of three replicates per tissue.

FIG. 3 shows chromatin accessibility of Ihcb10 promoter sequence as measured by ATAC-seq. Displayed is two reps from leaf-tissue data which shows the two regions of increased accessibility between −499 and −99 base pairs upstream of the transcription start site.

FIG. 4 shows β-Glucuronidase (GUS) expression in T₁ maize tissues transformed with a construct comprising the Ihcb 10 promoter operably linked to a gene encoding GUS. Blue cells indicate presence of β-glucuronidase activity. A: V1 Leaf B: V5 Leaf Base C: R1 11^{th} Leaf D: V9 Crown Roots E: 22 Days after pollination (DAP) Embryo F: Anther.

FIG. 5 shows GUS expression in T₁ maize tissues transformed with a construct comprising the ris2 promoter operably linked to a gene encoding GUS. Blue cells indicate presence of β-glucorinidase activity. A: V1 Leaf B: V5 Leaf Base C: V5 Leaf Tip D: R1 11^{th} Leaf E: Anther F: V1 Primary Root.

FIG. 6 shows GUS expression in T₁ maize tissues transformed with a construct comprising the aa2m promoter operably linked to a gene encoding GUS. Blue cells indicate presence of β-glucorinidase activity. A: 11 DAP Embryo B: 22 DAP Embryo C: V5 Leaf Base D: V5 Leaf Tip E: V9 Crown Roots.

FIG. 7 shows GUS expression in T₁ maize tissues transformed with a construct comprising the clo2b promoter operably linked to a gene encoding GUS. Blue cells indicate presence of β-glucorinidase activity. A: 11 DAP Embryo B: 22 DAP Embryo C: 22 DAP Endosperm D: R1 11^{th} Leaf E: Anther F: V9 Crown Root.

FIG. 8 shows GUS expression in T₁ maize tissues transformed with a construct comprising the expb14 promoter operably linked to a gene encoding GUS. Blue cells indicate presence of β-glucorinidase activity. A: 22 DAP Embryo B: V1 Leaves Base C: V5 Leaf Base D: V5 Leaf Tip E: V9 4^{th} Internode F: V9 Crown Root.

FIG. 9 shows GUS expression in T₁ maize tissues transformed with a construct comprising the nas2 promoter operably linked to a gene encoding GUS. Blue cells indicate presence of β-glucorinidase activity. A: V1 Primary Root B: V1 Primary Roots C: V9 Crown Roots D: Brace Root E: V9 8^{th} Leaf Base F: 22 DAP Embryo.

FIG. 10 shows GUS expression in T₁ maize tissues transformed with a construct comprising Version 2 promoters operably linked to a gene encoding GUS. Blue cells indicate presence of β-glucorinidase activity. A: V1 Primary Root—ris2 minimized promoter B: V1 Primary Root—Ihcb10 minimized promoter C: V1 Primary Root—Ihcb 10 fragment and CaMV 35S core promoter D: V1 Pooled Leaves-ris2 minimized promoter E: V1 Pooled Leaves—lhcb10 minimized promoter F: V1 Pooled Leaves-Ihcb10 fragment and CaMV 35S core promoter.

### DETAILED DESCRIPTION

The goal was to identify tissue-specific promoters and demonstrate their efficacy using quantitative and qualitative assays. Promoters were identified through RNA sequencing data. The inventors relied on this measure of promoter performance since that feature predominantly acts transcriptionally. Identified promoters were cloned into vectors driving expression of the GUSPlus gene (Broothaerts et al. 2005). This enabled qualitative and quantitative analysis of promoter strength, tissue specificity and timing via qualitative staining with 5-bromo-4-chloro-3-indolyl glucuronide (X-Gluc) and quantitative expression measurements with 4-methylumbelliferyl-beta-D-glucuronide (MUG). Maize inbred line LH244 was chosen for transformation with the promoter test vectors due to its ease of transformation and genetic similarity to maize genetic model line, B73, though new techniques with morphogenic genes have opened up the potential of direct transformation into B73 (Lowe et al. 2016). Six promoters (and fragments thereof) were shown to have tissue-specific expression: ris2, Ihcb10, and clo2b each show expression exclusive to their designed tissues while nas2, aa2m, and expb14 showed some off-target expression as well, but may be useful in some circumstances.

The present invention provides constructs comprising a transgene operably linked to thel tissue-specific maize promoters provided herein. Methods of using the constructs to drive tissue-specific transgene expression in a maize plant and maize plants comprising the constructs are also provided.

In the present application, the inventors describe promoters that drive tissue-specific gene expression in maize. By analyzing maize gene expression data, the inventors identified maize promoters that drive embryo-specific, root-specific, anther-specific, and leaf-specific gene expression. The functionality of these promoters was confirmed by transforming constructs comprising each promoter operably linked to a reporter gene into maize and analyzing reporter gene expression. These promoters expand the genetic tools available for driving tissue-specific transgene expression in maize and other plants.

### Constructs:

In a first aspect, the present invention provides constructs comprising a promoter operably linked to a transgene. The promoter included in the constructs is the promoter of a maize gene selected from the group consisting of ris2, Ihcb10, php20719a (clo2b), nas2, aa2m, and expb14 and fragments or portions of the upstream region of these genes capable of driving expression of transgenes. These promoter regions are capable of driving tissue-specific expression of the transgenes. The php20719a promoter is equivalent to the clo2b promoter, and these terms are used interchangeably herein.

As used herein, the term “construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) synthetic. Constructs can be generated using conventional recombinant DNA methods.

In some embodiments, the construct is a vector. The term “vector”, as used herein, refers to a polynucleotide that is capable of transporting another polynucleotide to which it is linked. Some vectors are capable of autonomous replication in a host cell into which they are introduced (e.g., bacterial vectors, such as plasmids, that include a bacterial origin of replication and episomal mammalian vectors). Other vectors can be integrated into the genome of a host cell such that they are replicated along with the host genome (e.g., viral vectors and transposons). Vectors may carry heterogeneous genetic elements that are necessary for propagation of the vector or for expression of an encoded gene product. Vectors may also carry a selectable marker gene, i.e., a gene that confers a selective advantage to a host organism, such as resistance to a drug or chemical. Suitable vectors include plasmids (i.e., circular double-stranded DNA molecules) and mini-chromosomes.

In some embodiments, the vector is an Agrobacterium vector. An “Agrobacterium vector” is a vector from the bacteria Agrobacterium tumefaciens that is used to insert foreign DNA into the genome of plant cells to generate transgenic plants. Agrobacterium vectors are derived from naturally occurring tumor-inducing (Ti) plasmids. Agrobacterium transfer a region of the Ti-plasmid known as the transfer DNA (T-DNA) into plants, where it is integrated into the host genome. The T-DNA is delineated by flanking it with T-DNA border repeat sequences. Thus, in some embodiments, the promoter and the transgene are inserted between T-DNA border repeat sequences in an Agrobacterium vector.

In other embodiments, the construct is a DNA donor template for CRISPR-mediated homology-directed repair (HDR). A “donor template” is a construct that comprises a sequence for insertion via HDR flanked by segments of DNA that are homologous to the ends of CRISPR cut sites. See the section titled “Methods” below for a more detailed description of CRISPR-mediated HDR. Those of skill in the art are capable of designing gRNAs and donor template for incorporation of DNA segments in a knock-in scenario.

A “promoter” is a DNA sequence that defines where transcription of a gene begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. A promoter is “operably linked” to a gene if the promoter is positioned such that it can affect transcription of the gene to which it is operably linked.

In the Examples, the inventors utilized ˜2 kilobase (kb) regions found upstream of the translation initiation site of the native maize genes ris2, Ihcb10, php20719a (clo2b), nas2, aa2m, and expb14 as promoters. The inventors simply modified about 3-4 nucleotides in each of these native sequences to make them suitable for golden gate cloning. Thus, in some embodiments, the constructs comprise or consist of the promoter sequences used by the inventors, i.e., SEQ ID NO: 1 (php20719a), SEQ ID NO: 2 (ris2), SEQ ID NO: 3 (lhcb10), SEQ ID NO: 4 (nas2), SEQ ID NO: 5 (aa2m), or SEQ ID NO: 6 (expb14). In other embodiments, the constructs comprise a promoter that shares at least 75%, at least 80%, at least 85%, at least 90%, at least 95%, at least 97%, at least 98%, or at least 99% sequence identity with the ˜2 kb native sequence or the modified sequence used by the inventors. In other embodiments, the promoter comprises only a portion of the ˜2 kb native sequence or the modified sequence used by the inventors. In these embodiments, the portion of the sequence is preferably a “functional fragment,” i.e., a fragment that is capable of functioning as a promoter, but less than the full-length sequences of SEQ ID NO: 1-6. In the examples the inventors demonstrate the utility of the minimal promoters for some of these sequences, e.g. (SEQ ID NO: 7 (ris2-min), SEQ ID NO: 8 (lhcb10-min). Those of skill in the art will understand that the same functional fragments can be generated for the remaining promoters provided herein. These fragments include at least 100 bp, 200 bp, 400 bp, 500 bp, 750 bp, 1 kb or more of the 2 kb segments used in the examples.

“Percentage of sequence identity” or “percentage of sequence similarity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc. Natl. Acad. Sci. USA (1990) 87:2267-2268; Nucl. Acids Res. (1997) 25:3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula Proc. Natl. Acad. Sci. USA (1990) 87:2267-2268), the disclosure of which is incorporated by reference in its entirety. The BLAST programs can be used with the default parameters or with modified parameters provided by the user.

As used herein, the term “transgene” refers to genetic material (e.g., a gene or portion thereof) that one wishes to transfer into an organism or cell. In some embodiments, the transgene encodes a reporter, a pharmaceutical, or a nutrient. In some embodiments, the transgene encodes a protein that (a) confers resistance to an infection (e.g., by a bacterium, fungus, or virus), an insect, an herbicide, drought, waterlogging, or high temperatures; or (b) confers an increase in maize yield or nutritional content.

As used herein, a “reporter” is a molecule that creates a detectable signal. Suitable detectable signals that can be produced by a reporter include, without limitation, fluorescent signals, luminescent signals, colorimetric signals, wavelength absorbances, and radioactive signals. In the Examples, the inventors linked their candidate tissue-specific promoters to the reporter β-glucuronidase (GUS). GUS is an enzyme that converts specific colorless or non-fluorescent substrates into stable colored or fluorescent products. Thus, the use of this reporter allowed the inventors to determine whether their candidate promoters drive gene expression in a particular tissue based on the presence or absence of GUS-induced color or fluorescence in that tissue.

The promoters provided herein provided for differential expression levels of a transgene when operably linked to a transgene and for tissue specific expression of the transgene. If embryo-specific expression is needed the promoters used may include the aa2m, clo2b, or expb14 promoter or a fragment of any one of these promoters. If root-specific expression is needed, the nas2 promoter can be used or a fragment of the nas2 promoter. If anther-specific or leaf-specific expression is needed, the Ihcb10 or ris2 promoters or fragments of one of these promoters may be used.

### Methods:

In a second aspect, the present invention provides methods for driving expression of a transgene in a maize plant. The expression of the transgene may be tissue-specific expression. The methods comprise introducing a construct described herein (i.e., a construct comprising a promoter operably linked to a transgene) into the maize plant.

As used herein, the phrase “tissue-specific expression” refers to gene expression that is restricted to a particular tissue or group of tissues or a particular cell type or group of cell types. The tissue specific expression may also refer to expression in particular tissues at particular developmental stages. As is described in the Examples, the inventors discovered promoters that can be used to drive embryo-specific expression, root-specific expression, or leaf-specific expression. Thus, in some embodiments, the tissue-specific expression is embryo-specific, root-specific, or leaf-specific. As noted above, the inventors have demonstrated that certain of the promoters provided herein provided tissue-specific or cell-type specific expression of transgenes to which the promoters are operably connected.

As used herein, “introducing” describes a process by which exogenous polynucleotides are introduced into a recipient cell. Suitable introduction methods include, without limitation, Agrobacterium-mediated transformation, the floral dip method, bacteriophage or viral infection, electroporation, heat shock, lipofection, microinjection, and particle bombardment.

In some embodiments, the construct is introduced via Agrobacterium-mediated transformation. In this method, the construct is delivered into plant cells as part of a binary Agrobacterium vector, in which the construct is flanked by two T-DNA repeats. Prior to transformation into plant cells, this binary vector is co-transformed with a second vector, referred to as a vir helper plasmid, into Agrobacterium tumefaciens. The vir helper plasmid encodes components necessary for integration of the region flanked by the T-DNA repeats into the genome of plant cells. Thus, when the binary vector and the vir helper plasmid are both present in the same Agrobacterium cell, proteins encoded by the vir helper plasmid act in trans on the T-DNA border repeat elements to mediate processing, secretion, and host genome integration of the intervening transgene. Genome insertion occurs without any significant bias with respect to insertion site sequence.

In other embodiments, the construct is introduced via CRISPR-mediated homology-directed repair. “Homology directed repair (HDR)” is a naturally occurring nucleic acid repair system that is initiated by the presence of double strand breaks (DSBs) in DNA. In CRISPR-mediated HDR, CRISPR is used to create targeted DSBs (i.e., by targeting a nuclease to cut at specific loci using guide RNAs that are complementary to those loci), which are then repaired using a donor template. The donor template comprises a sequence for insertion flanked by segments of DNA that are homologous to the ends of the DSBs. Thus, in cells that repair the DSBs using the donor template, the genome will be edited to include the sequence for insertion between the sites of the DSBs. Any form of donor template known in the art may be used in the methods of the present invention, including single-stranded oligodeoxynucleotides (ssODNs) and donor plasmids.

In some embodiments, the construct is incorporated into the genome of the maize plant, whereas, in other embodiments, the construct is maintained as extrachromosomal DNA (i.e., DNA that exists outside of the chromosomes that make up the plant genome).

### Plants:

In a third aspect, the present invention provides transgenic or genetically engineered maize plants comprising a construct described herein. As used here, a plant is “transgenic” if the plant comprises one or more cells that comprise a transgene. In some embodiments, the transgene is incorporated into the genome of the maize plant, whereas, in other embodiments, the transgene is maintained extrachromosomally. In still other embodiments, the promoters described herein may be used to replace the native promoter driving expression of a native gene using homologous recombination, CRISPR-mediated gene editing or other means available to those of skill in the art to genetically engineer plants or plant cells.

In some embodiments, the transgenic or genetically engineered maize plant was produced via a method described herein and comprises one of the promoters provided herein operably linked to a non-native gene to allow for tissue-specific or cell-type specific expression of the linked gene.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

In the following example, the inventors describe the identification and validation of four novel tissue-specific maize promoters.

### Materials and Methods:

### RNA Seq Data Collection, Analysis, and Comparison

RNA was extracted from maize plant tissues using the Monarch Total RNA kit (New England BioLabs catalog #T2010S) with DNase treatment. RNA QC was done on the Agilent PicoChip (Agilent Technologies item #50671513). Each library was created with the TruSeq Stranded mRNA Kit (Illumina item #20020595). Libraries were quantified in singlet on a Qubit with dsDNA HS reagent. Libraries were sequenced on a NovaSeq 6000. Paired-end, 150 bp sequencing was performed. Data was processed with bcl2fastq. Sequencing was provided by the University of Wisconsin—Madison Biotechnology Center's DNA Sequencing Facility (Research Resource Identifier—RRID: SCR_017759). Alignment and TPM calculations were done using Salmon (Patro et al. 2017). Principal component analysis and Pearson correlations were calculated with R (R-Core-Team 2023).

### Identification of Tissue-Specific Promoter Candidates

Candidate promoter sequences were identified from version 4 of the maize genome sequence (Jiao et al. 2017). Sequences were filtered to remove promoters with long-range activating cis-regulatory elements, which can be tens of thousands of bases away and are not captured readily by cloning (Lu et al. 2018; Ricci et al. 2019). Additionally, only named genes on MaizeGDB were considered to ensure some level of documentation about the gene. RNA-seq expression data from the maize gene atlas, which contains expression data from all major tissues in maize, was utilized to identify genes with a desired expression profile (Sekhon et al. 2013; Stelpflug et al. 2016). Expression was normalized to “transcripts per million” (TPM). ZmUbi1 expression in tissues was used as the standard of high, constitutive expression to create an idealized expression level within the tissues targeted. Specifically, maize zygotic embryos, leaves, and root tissues were targeted for promoter identification. Candidates were ranked by their Euclidean distance to the idealized expression level. High ranking candidates were manually appraised for their utility and genes that seemed influenced by stress or environmental conditions were eliminated from consideration.

### Cloning of Candidate Promoter Sequences

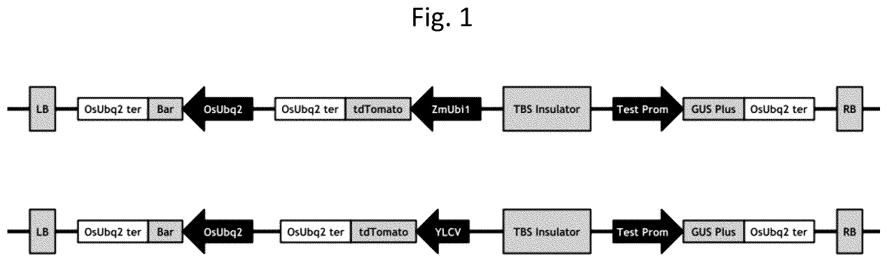

Sequences for identified promoters were isolated from the version 4 sequence of the maize genome (Jiao et al. 2017). The first 2 kb of promoter sequence, including any annotated 5′-UTR, was synthesized as a level 0 compatible part for Golden Gate cloning which was used to drive GUSPlus expression (Broothaerts et al. 2005; Engler et al. 2014). The Osl/bq2 promoter was used to drive the bar gene (Thompson et al. 1987; Wang et al. 2000b). The red fluorescent protein reporter gene, tdTomato, was expressed with either the ZmUbi1 promoter or the YLCV promoter (Christensen et al. 1992; Stavolone et al. 2003). The Bar and tdTomato expression cassettes were separated from the tested promoter by the TBS insulator (Hily et al. 2009). Two versions of the transformation vector were created, with the only difference being in the promoter utilized to drive tdTomato expression (Shaner et al. 2004). In version 1, the promoter used was Zmlbil and in version 2 the Yellow Leaf Curling Virus promoter was used (Christensen et al. 1992; Stavolone et al. 2003). All enzymes used were sourced from New England Biolabs. A linear map of the T-DNA portion of the binary vector is provided in FIG. 1. Shortened versions of ris2 and Ihcb10 promoters were created by visually analyzing ATAC-seq data collected from young leaves (Ricci et al. 2019). The shorted versions of ris2 and Ihcb10 are indicated herein as ris2-min and Ihcb10-min, respectively. Cutoffs were assigned based on where accessible chromatin ended.

### Stereo Microscope Imaging

A Leica stereomicroscope (model M165 FC) with a DFC 7000T camera attached was used to take all sample photographs with the LAS X software suite.

### Plant Materials

Carlin SVD-250 pots were arranged in Carlin 3-234 ST-I-0804 vacuum standard inserts. A custom potting mix based on “Jolly Gardener” brand Pro-Line C/B (Part No. 18-1010) was distributed to the pots. Seeds of maize inbred line, LH244, were sown by placing one seed per cell, pressing down ¾ inch to 1 inch, then covering with loose, dry soil medium. Flats were placed on benches and manually watered until day 7 to 10, when flood fertigation was begun. Seedlings were grown in flats in the nursery area of the greenhouse for 18 to 21 days. Greenhouse rooms were illuminated with high pressure sodium lamp fixtures (model #HS 2000 from PL lighting) with 600 W Phillips E39 HPS bulbs and supplemented with natural sunlight. The light fixtures were mounted about 2.7 m above the floor. Seeds were germinated on a bench top approximately 1.7 m below the lights. Custom LED fixtures were used for supplemental lighting. The LED fixtures were 600 W and 1.3 m from the bench top. Average photosynthetic photon flux density (PPFD) was 500 μmol m-2 s-1 on the bench top but varied between 370 and 660 μmol m-2 s-1 depending on the age of fixtures, the time of day and time of year. After 18 to 21 days, plants were transferred to size 1200 Elite Blow-molded Nursery Containers (Carlin, Inc. catalog #4-2022). The potting mix was the same as above. Pots were placed into carts, with the cart surface approximately 1.7 m below the light fixtures. After 7 to 9 days, pots were moved to the floor and placed on plastic pallets.

The temperature set points for the greenhouse were 26.7° C. during the day and 21.1° C. during the night. The photoperiod consisted of a 16-hr light: 8-hr dark with supplemental sunlight during the light period. The fertigation mix utilized was Peters Excel 15-5-15 Cal Mag Special (Carlin item number 20-235). Plants were on timed fertigation cycles based on growth stage and demand.

### Sampling of Plant Materials

Table 1 indicates the stages of growth and tissues assayed for gene expression.

TABLE 1
Stages of growth and tissues assayed for gene expression

| Stage | Tissues |
|---|---|
| V1 | Stem & shoot apical meristem, 1^{st} leaf, primary root |
| V5 | Daytime base of 6^{th} leaf, daytime tip of 6^{th} leaf, night |
| | base of 6^{th} leaf, night tip of 6^{th} leaf |
| V9 | 8^{th} leaf, 4^{th} internode, nodal roots from 3^{rd} node |
| Reproductive | 11^{th} leaf, brace roots, anthers, 2 days after pollination |
| | (DAP) seeds, 11 DAP embryos, 22 DAP embryos, |
| | 22 DAP endosperm |

V1 Plant Sampling: Primary Root: A 2 cm piece of primary root was collected, measuring from the base of the roots. Stem & Shoot: Plants were cut just above the root growth point and 2 cm up the stem. A twisting motion with the fingertips was used to remove the other leaves resulting in the meristem being exposed. Pooled Leaves: All leaf material 2 cm above the base of the plant was collected. V5 Sampling: Only plants where the ligule of the 5th leaf had emerged less than an inch were sampled. 6th Leaf Base: The stem was cut from the roots and the outer 5 leaves were pealed away. A 1 cm section just above the faint ligule of the 6th leaf was sampled. 6th Leaf Tip: A 2 cm long segment of the 6th leaf was sampled.

V9 Sampling: 8th Leaf: The 8th leaf was isolated a cut 2 cm above the ligule and 1 cm below it were made to create the sample. 4th Internode: The sample was taken by cutting just below the 5th node and just above the 4th node. Crown Roots: Three crown roots from the 3rd node were collected from the plant and a 2 cm section was sampled 10 cm away from the where the root grew from the node.

R1 Sampling: Brace Roots: A 2 cm section was cut from the tip of brace roots from the 5th node of the plant. 11th Leaf: 30 cm from the tip of the 11th leaf, a 2 cm wide segment of the leaf was sampled. Anthers: Four sets of anthers were taken from the main tassel just below where pollen shed had begun.

### Genetic Transformation of Maize

Test vectors were transformed into maize inbred LH244 using Type I embryonic callus induction. The protocol utilized was originally developed for maize inbred B104, but, with minor modifications also worked for LH244 (Kang et al. 2022). Agrobacterium tumefaciens strain AGLI was used for all transformations (Lazo et al. 1991). Single copy events were identified by examination of segregation ratios of tdTomato expression in T1 seeds. Sixty seeds were assayed per event and those with 33 positive seeds or less were deemed single copy for falling within the expected 1:1 segregation ratio for transgenic to non-transgenic progeny from a transgenic TO by null plant cross.

### Gus Staining Protocol

A buffered solution of X-gluc was used to assess expression and localization of transgene-encoded beta-glucuronidase (GUS) at a qualitative level (Hackett 1993). The buffer was a 0.1M solution of K2HPO4 dipotassium phosphate, brought to a pH of 7 with HCl/NaOH. Triton x-100 was then added to create a final concentration of 0.5%. The buffer was stored at 4° C. until used. To create the final GUS assay solution, X-gluc dissolved in DMSO (at 50 mg/mL) was added to the buffer solution to a final concentration of 100 mg/L. Material to be assayed was dissected from plants as specified above and immediately submerged in the solution. Three rounds of applied vacuum were used to infiltrate the solution into the tissues. The tissues were stained overnight for 16 hours at 37° C. Samples were cleared of chlorophyll using 100% ethanol and stored in 70% ethanol.

### MUG Assay Protocols

MUG assays were performed as standard procedure (Hackett 1993). Samples were stored at 2° C., never frozen, for up to 24 hours as needed. Fluorescence measurements were performed on a Tecan SPECTRAFluor Plus microplate reader.

### Results:

Identification, Isolation, and Cloning of Candidate Tissue-Specific Promoters Candidate promoters were identified from the pool of maize tissue expression data in the Maize Gene Atlas (Sekhon et al. 2011, 2013; Stelpflug et al. 2016). Tissue-specific expression was identified by looking for genes with high expression levels in desired tissue and low off-target expression, ranking candidates based on their Euclidean distance from an “ideal expression profile”. Manual curation of candidates resulted in 14 promoter sequences being identified that appeared to drive leaf-, seed-, or root-specific gene expression. The manually curated list of promoter candidates can be found in Table 2. The gene name from the fourth version of the maize genome sequence as well as the common name are provided.

TABLE 2
Identity of genes assayed for tissue-specific expression

| Vector ID | V4 Gene Name | Common Name | Tissue | Genomic Coordinates (V4) |
|---|---|---|---|---|
| NSV049 | Zm00001d011467 | csu31a | Leaf | chr8: 150719482-150720472 |
| NSV050 | Zm00001d044666 | fha9 | Leaf | chr3: 236263686-236264888 |
| NSV051 | Zm00001d037513 | glp1 | Leaf | chr6: 134533295-134534385 |
| NSV052 | Zm00001d011285 | lhcb10 | Leaf | chr8: 145361964-145363001 |
| NSV053 | Zm00001d053432 | ris2 | Leaf | chr4: 234647110-234648701 |
| NSV078 | Zm00001d020590 | aa2m | Seed | chr7: 125329517-125334159 |
| NSV079 | Zm00001d037436 | dzs18 | Seed | chr6: 132167487-132168306 |
| NSV080 | Zm00001d045792 | expb14 | Seed | chr9: 41286339-41287799 |
| NSV081 | Zm00001d009292 | fl3 | Seed | chr8: 53009968-53011139 |
| NSV082 | Zm00001d034413 | glb2 | Seed | chr1: 293871724-293873694 |
| NSV083 | Zm00001d002768 | ole | Seed | chr2: 22219327-22220208 |
| NSV084 | Zm00001d025833 | php20719a | Seed | chr10: 132695374-132697286 |
| NSV085 | Zm00001d028888 | nas2 | Root | chr1: 49167559-49169755 |
| NSV086 | Zm00001d026177 | por2 | Root | chr10: 141833690-141835020 |

Identified promoters were synthesized based on their sequence in version 4 of the B73 genome, with the first 2 kb upstream sequence cloned along with any 5′-UTR if present (Jiao et al. 2017). The promoters to be tested were placed in front of GUSPlus (Brothaerts et al. 2005) gene sequence to drive expression. For comparison, a T-DNA containing the ZmUbi1 strong, constitutive promoter driving GUSPlus was also created (Christensen et al. 1992). T-DNA expression vectors were constructed using Golden Gate cloning and included the tdTomato gene under the control of the ZmUbi1 or the CmYLCV promoter to visually track events and the bar gene (providing resistance to the herbicide, bialophos) driven by the OsUbq2 promoter to enable selection of transgenic cells/plants (Thompson et al. 1987; Christensen et al. 1992; Wang et al. 2000a; Stavolone et al. 2003; Engler et al. 2014). Promoter testing transcriptional units were separated from other transcriptional units within the T-DNA region of the vector by the petunia transformation boost sequence (TBS) insulator. This insulator has been shown to block enhancing activity it separates in Arabidopsis thaliana and Nicotiana tabacum, though some studies have shown it can mildly enhance expression and might not reduce variability (Hily et al. 2009; Singer et al. 2011; Dietz-Pfeilstetter et al. 2016; Pérez-González and Caro 2019). All candidate promoters were successfully cloned into expression vectors (FIG. 1). Transformation treatments were successful with all promoters in LH244 and at least 3 independent single-copy events were generated for each promoter, taken from a larger generated pool of events.

### Assessment of LH244 Transcriptome Compared to B73

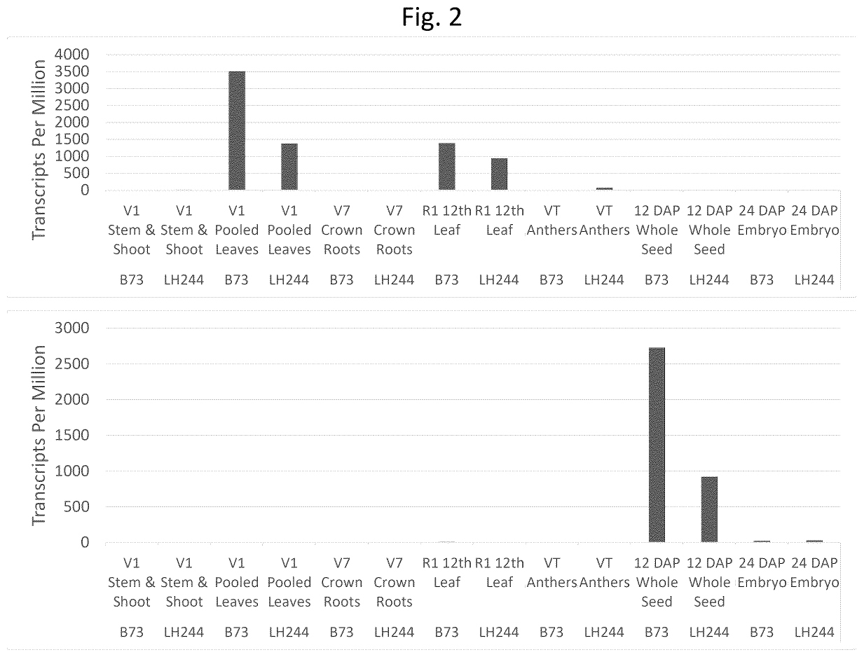

RNA-sequencing data was successfully generated from a reduced set of tissues from the B73-based Gene Atlas, specifically, in 11 “days after pollination (DAP) whole seeds, 24 DAP embryos, V1 pooled leaves, R1 12^{th} leaf, V1 stem & shoot, V7 crown roots, and VT anthers (Abendroth et al. 2011). Generated reads were aligned to the B73 version 5 transcriptome alongside reads from the Maize Gene Atlas (Hufford et al. 2021). Initial comparison was conducted using principal component analysis on the “transcripts per million” for each gene which did not result in like-tissue samples grouped with one another but instead were separated by their variety. This lack of grouping was further confirmed by Pearson correlations between the samples, which ranged from 0.43 to 0.79 with “V7 crown roots” and “R2 12th Leaf” comparing the worst (David Freedman, Robert Pisani 2007). The pattern of expression of candidate promoters was checked visually between the two varieties, with parity found between each in their tissue specificity. Two example promoter profiles are shown in FIG. 2.

### Qualitative and Quantitative Assessment of Promoters for Tissue-Specificity

Testing of promoters for strength and specificity necessitated development of a set of tissues that would be sufficient for capturing potential off target expression while minimizing the number of tissues collected. Principal component analysis was performed on RNA sequencing data, again from the Maize Gene Atlas, to identify which tissues separate based on expression. This resulted in 14 tissues being identified to cover the whole development cycle of maize (Table 2). Initial assessment was conducted on T0 plant tissues which were stained with X-Gluc in the targeted tissues with further investigation conducted on those promoters which showed promise. Subsequent investigation was done on T1 plants which were hemizygous for the transgene and single copy. Tissue samples from the growing plants were taken at each phase listed in Table 1 and stained with X-Gluc. Additionally, V1 leaves and primary roots from Ihcb10 and ris2 were measured for GUS activity through a MUG assay. The results of the MUG assay can be found in Table 3.

TABLE 3
MUG Assay Results to Determine Expression Levels of
Candidate Promoters in Maize Leaf and Root Tissues

| V4 Gene Name | Tissue | Event 1 | Event 2 | Event 3 |
|---|---|---|---|---|
| lhcb10 | Leaf | 33.31* | 105.86* | 78.34* |
| | Root | 3.75 | 3.95 | 4.44 |
| ris2 | Leaf | 4.58 | 6.73* | — |
| | Root | 2.77 | 3.98 | — |
| lhcb10-min | Leaf | 30.64* | 73.64* | 4.03* |
| | Root | 3.21 | 3.11 | 3.91 |
| ris2-min | Leaf | 7.91* | 8.76* | 7.16* |
| | Root | 2.72* | 2.85 | 4.07 |
| lhcb10-syn | Leaf | 3.77* | 5.98 | 6.24* |
| | Root | 2.48* | 1.72 | 3.16 |
| ubi1 | Leaf | 87.24* | | |
| | Root | 48.26* | | |
| LH244 Wild Type | Leaf | 1.51* | | |
| | Root | 4.38* | | |

Table 3. MUG assay results to determine expression levels of candidate promoters in maize leaf and root tissues.
“lhcb10-min”: shortened lhcb10 promoter.
“ris2-min”: shortened ris2 promoter.
“lhcb10-syn”: conjugate promoter created with the lhcb10 fragment with the CaMV35S promoter.
All measurements are in units of nmol 4-MU min − 1 mg − 1.
All measurements shown are an average of three replicates per event.
*The sample is significantly different in MUG activity compared to the LH244 control samples, which have a mean of 4.38 for root tissue and 1.51 for leaf tissue.

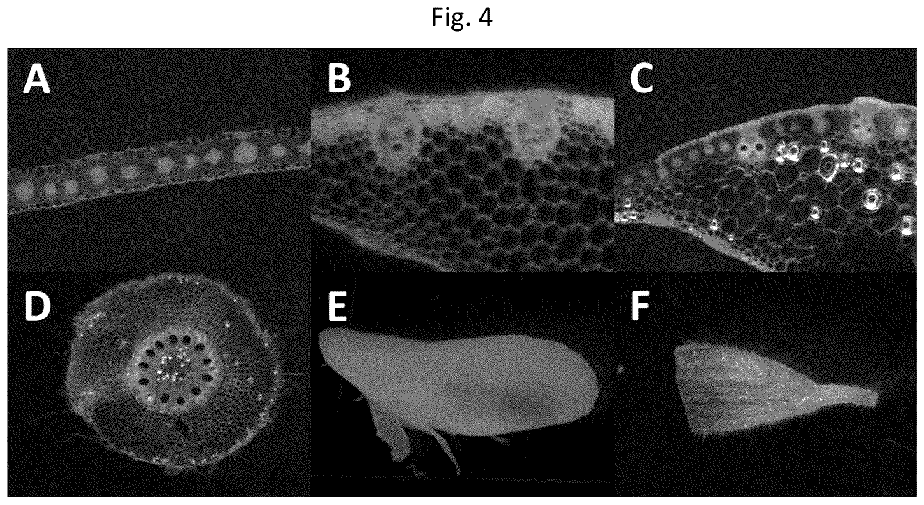

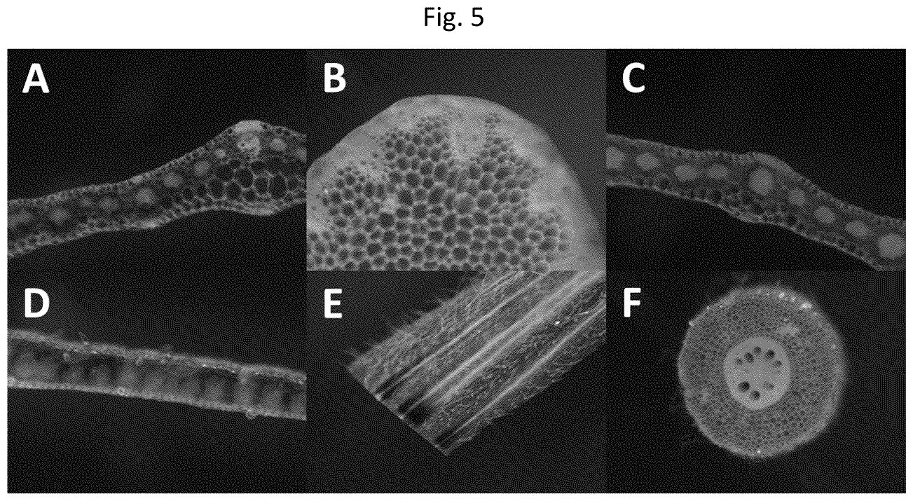

Among the leaf samples, only lhcb10 and ris2 showed visible GUS staining results. Examples of visual GUS assay results for the lhcb10 promoter and ris2 promoter are shown in FIG. 4 and FIG. 5, respectively. Both showed consistent staining specifically in the mesophyll tissue of leaves across the entire life cycle of the plant. The only off-target expression detected was in the green tissue of anthers. MUG assay results for the two promoters confirm the visible GUS assay results for activity level and show that Ihcb10 is a much stronger promoter than ris2 ( ).

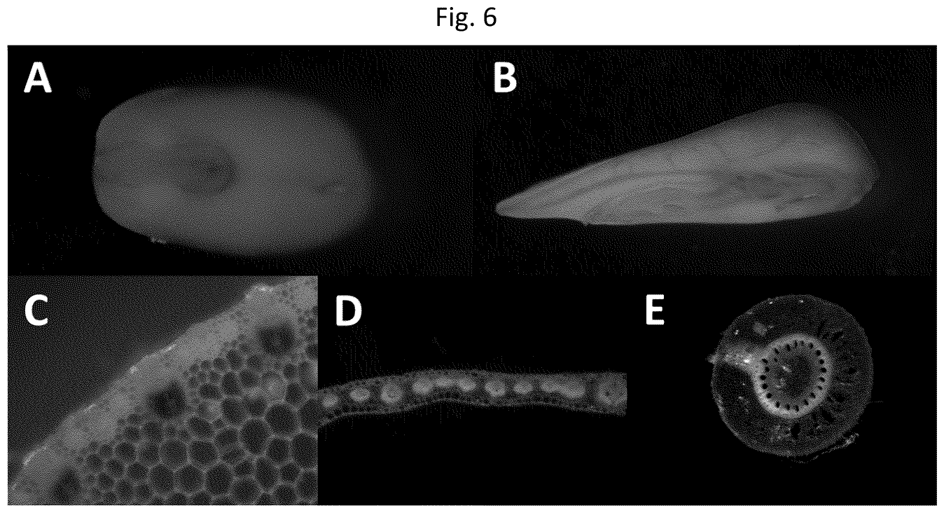

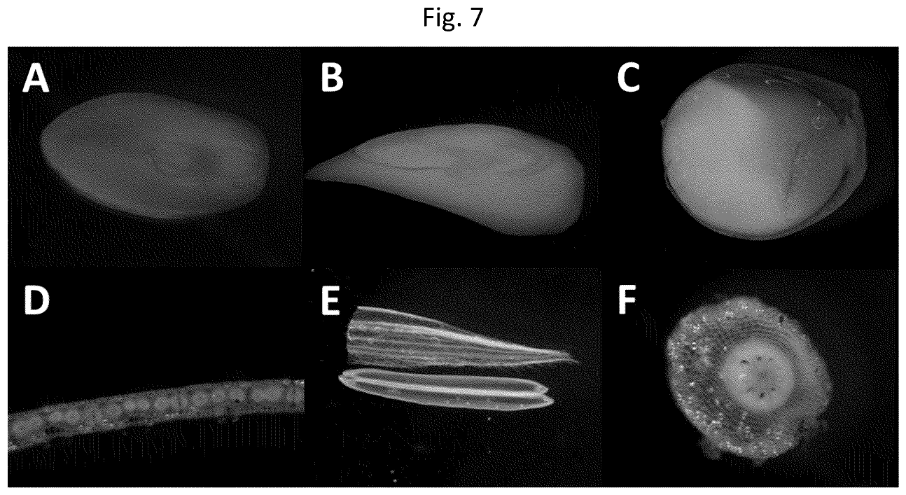

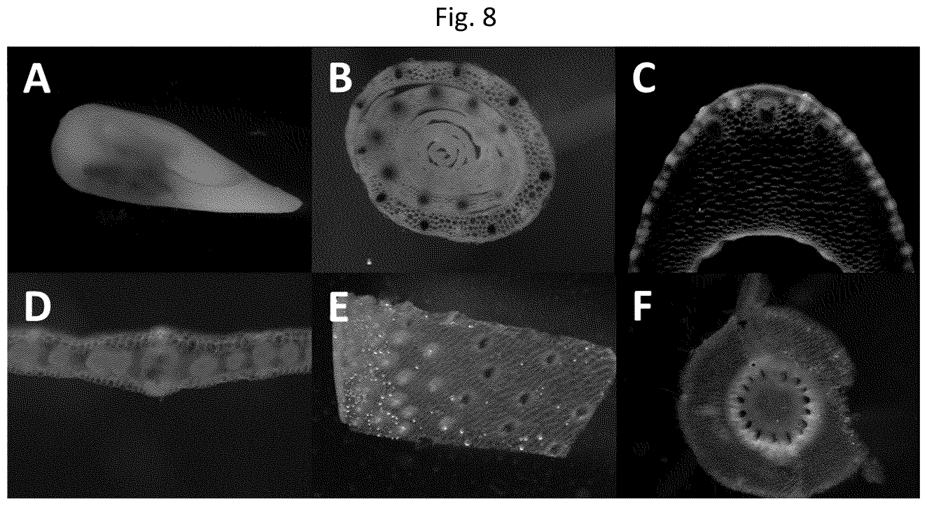

Three embryo-specific promoters were identified: aa2m (FIG. 6), clo2b (FIG. 7), and expb14 (FIG. 8). The aa2m promoter was most active in the 11 DAP embryo though still showed activity/staining at 24 DAP, most prevalently along the scutellar surface. It was also very active in the leaf vasculature at the base of the 6th leaf from the V5 growth phase. There was no activity detected elsewhere including the tip of the V5 6th leaf. The clo2b promoter displayed high activity in the 11 DAP embryo, but was very faint by 24 DAP, with only some GUS staining occurring in the aleurone. No other GUS expression was detected in any other tissues tested. Finally, the expb14 promoter was shown via GUS assay to be active in the 24 DAP embryo, within the scutellum itself and at its juncture with the nascent plant. Staining was also strongly visible within the still-growing vasculature of above-ground tissues, present in the V1 leaves, V5 6th leaf base, and the fourth internode. Expression faded within mature tissue such as the tip of the V5 6th leaf. No staining was observed in root tissues.

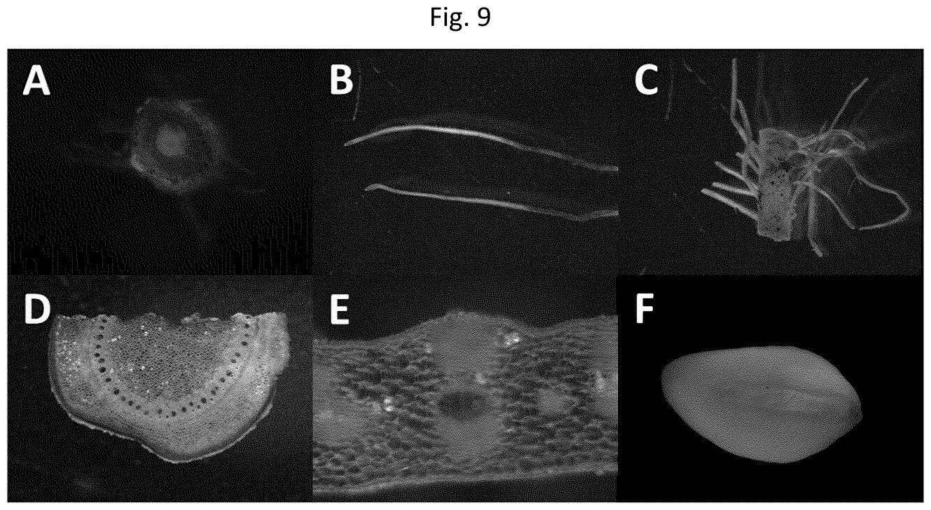

Only one root-specific promoter was identified, nas2 (FIG. 9). Visual GUS assays demonstrated strong nas2-driven GUS expression in the primary root, specifically in the cortex. The promoter was shown to be active in mature root tissue and did not appear in the elongation zone or at the root cap. In crown roots, GUS expression was detected in the secondary root, though much weaker compared to the primary root. Expression was not observed in brace roots or any reproductive tissues. The nas2 promoter was also found to be active in the vasculature specifically in the V9 8th leaf, which was sampled close to the ligule. Expression was not detected elsewhere on the leaf such as the V1 leaves or the V5 6th leaf.

### Generation and Assessment of Minimal Promoter Sequences and Synthetic Promoters

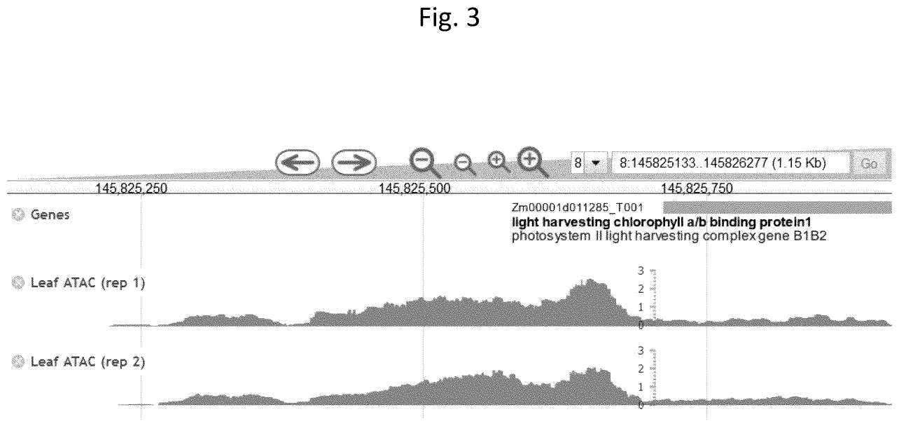

The Ihcb10 and ris2 promoters were reduced from their original 2 kb size to 499 bp and 400 bp, respectively, based on chromatin accessibility data in maize leaf (Ricci et al. 2019). In addition, a fragment of Lhcb10 was isolated based on accessible chromatin from leaf tissue (Ricci et al. 2019). A 400 bp segment from −499 to −99 from the transcription start site in the B73 V4 genome was selected (FIG. 3). However, in the V5 annotation for maize, this fragment corresponds with −395 to +4 of the transcription start site as a 5′UTR had been added compared to the V4 version. This fragment was combined with CaMV35S and ZmUbi1 core promoter sequences to construct a complete promoter. The core sequences were also cloned on their own into a T-DNA expression cassette as a control. All the promoters were synthesized then cloned into T-DNA expression cassettes like previous constructs (FIG. 1) and transformed into LH244.

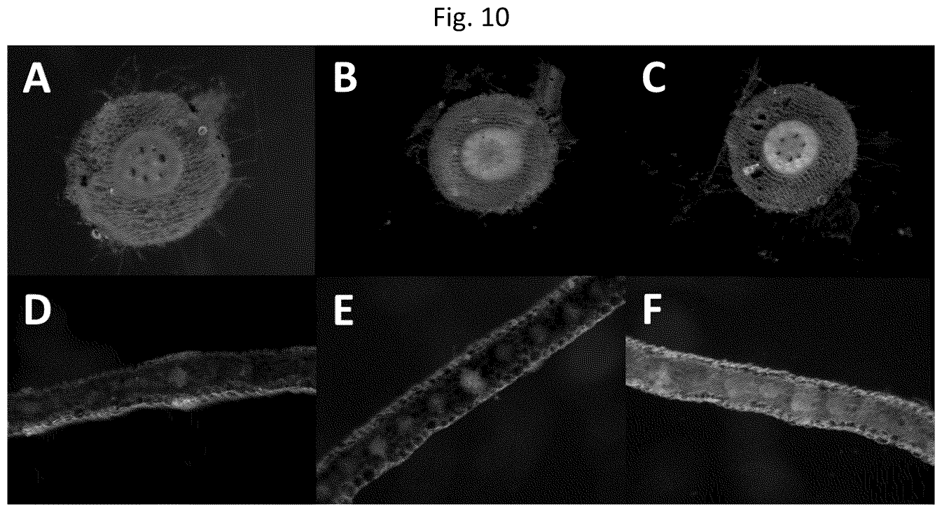

GUS staining results indicated activity of both minimal promoters exclusively in the shoot but not the root (FIG. 10). MUG assay results were like the visual assay in that that both minimal promoters maintained their rank relative to one another in strength and their strength compared to the longer versions (Table 3). Neither core promoter showed GUS staining in any of the tissues sampled. The Ihcb 10 fragment with the ZmUbi1-core sequence also did not demonstrate positive GUS staining in any tissues. The fragment combined with CaMV35S-core showed positive staining in the leaves, but none in roots (FIG. 10). Results from MUG assays indicated that the expression level of the Ihcb 10 fragment with the ZmUbi1-core sequence was greatly decreased relative to either the full length or minimized promoter sequence.

### Discussion:

### Maize Inbred LH244 as a Transformation Platform

Analysis of the LH244 expression data within the context of B73 reveals the difficulty in experiment duplication and the incomplete picture genomic data provides. While B73 and LH244 are very similar genetically with 97.1% parity in SNPs, clear expression differences between the two were observed, specifically in the reproductive-phase leaf and crown root tissues. LH244's genomic background is closer to that of elite lines and may explain some of the differences observed specifically in the reproductive phase leaf and crown roots. An additional difference might be in sampling practices and conditions, despite best efforts to prevent these. Ultimately, the expression profiles of candidate promoters are closely aligned between B73 and LH244, demonstrating its utility for this research's goals.

Leaf Promoter Performance: Ihcb10 and Ris2 and their Variants

Light harvesting chlorophyll a/b binding protein 1 is a nuclear encoded component of the photosystem II (Bansal and Bogoradt 1993; Rocca et al. 2000). Given its central role in photosynthesis, it has been identified in many trait assessments including stalk biomass, anther development, and drought resistance (Mazaheri et al. 2019; Li et al. 2021; Han et al. 2022). Transgene-based expression studies of the promoter in the maize variety FR9cms x FR37 showed that the promoter provided mesophyll-specific activity which is specifically mediated by a 159 bp region at −1026 to −868 upstream of the transcription start site, studied through particle bombardment of leaf material (Bansal and Bogoradt 1993). This corresponds with the GUS staining observations of lhcb10, which is specific to the mesophyll and absent in bundle sheath cells. Our observations, however, did not match this previous study with our minimized Ihcb10 sequence which excluded these indicated regions and thus should have shown a decrease in overall expression plus an appearance of bundle sheath expression, neither of which is shown. A possible explanation for this is a difference of approach, as the original study utilized particle bombardment-based transient expression assays while we worked with stably transformed events. Additionally, we were working in a different maize genotype whose expression network might be influencing the promoter's activity. Rieske iron-sulfur protein 2, ris2, is a much more straightforward success in terms of identifying a promoter with tissue-specific activity. In maize, the RIS2 protein is a component of the cytochrome f/b6 complex, which participates in the electron transport chain (Barkan and Walker 1994; Klusch et al. 2022). It is known to be involved in photosynthesis, and its expression in leaves is expected (Simkin et al. 2017). The main potential detraction of the promoter is its weak performance compared to Ihcb10 and ZmUbi1, but this might be useful in situations where low but present expression is necessary.

Utilization of the first 2 kb of promoter sequences for our tests was a very conservative approach, as evidenced by the success of minimizing the promoter sequences of lhcb10 and ris2. Their smaller sequence size can increase their utility while minimizing their impact on T-DNA size. A natural limitation of this approach is that it necessitates having chromatin availability data in the specific tissue targeted. However, with the decreasing costs of next-generation sequencing, the available tissue information will only expand. Neither core promoter demonstrated expression alone, which is ideal when creating synthetic promoters built on core promoters. Results from tests of the final Ihcb10-synthetic promoters are difficult to interpret, given that the fragment isolated likely also includes the core promoter of Ihcb10. This core piece however does not seem sufficient for expression on its own, as if that was the case then both promoters would be expressed. However, the CamV35S core promoter combined with the Ihcb10 fragment was actually expressed.

Interpretation of Seed-Specific Expression of Promoters from Maize genes aa2m, clo2b, and expb14

Expression of alanine transferases has been observed in maize in two tissues, seed and leaf, both of which were observed in tests of the alanine transferase promoter sequence aa2m. Alanine transferases have been found in scutellar tissue of the embryo, where various isozymes were described (Watson et al. 1992). Presence of the enzyme matches the expression pattern observed for the GUS gene driven by the aa2m promoter, with strong GUS staining observed in scutellar tissues of transgenic maize embryos at 11 DAP and even into 24 DAP. Other studies have shown alanine transferase activity in the endosperm, however, we did not detect GUS expression in that tissue with the aa2m promoter (Faleiros et al. 1996). Within the leaf tissues, alanine transferase activity is noted to increase as the leaf matures, as it is involved in a secondary decarboxylation pathway (Pick et al. 2011; Lori Tausta et al. 2014). The activity of the aa2m promoter in our tests, based on our GUS expression assays did not fit that scheme, being expressed in the vasculature and not the bundle sheath or mesophyll cells. Further research may unveil the role and functions of aa2m in both embryo and vasculature, however, the characterized promoter may be useful for other purposes.

ZmClo2b, also known as ZmPhp20719a, is a member of the caleosin/peroxygenases family of proteins. Originally identified through desiccation studies, this family has been found to associate with lipid bodies as well as having catalytic functions (Yamaguchi-shinozaki et al. 1992; Chen et al. 1999; Næsted et al. 2000; Hernandez-Pinzon et al. 2001). This family is ubiquitous in the plant kingdom and, within maize, 12 caleosin genes have been identified (Lizong et al. 2014; Hanano et al. 2023). As a family, caleosin has been detected in the embryo of maize (Tnani et al. 2011). Specifically, for ZmClo2b, it has been detected through RT-PCR in seed material (Lizong et al. 2014). As a family, though, they are detected in many different tissues (Lizong et al. 2014). Furthermore, mutations in the gene have been linked to smaller and thinner kernels (Jia et al. 2016). The expression pattern observed in our tests of the promoter of this gene exclusively in the early embryo of maize perfectly aligns with each of these expectations. Notable is that the expression does not persist into 24 DAP, which makes sense given that embryo development has been slowed by that stage of seed growth.

The expansin family has an observed role in seed development and has been linked to seed yield in sunflowers, Arabidopsis, and other plants (Bae et al. 2014; Castillo et al. 2018). Specifically in maize, expb14 and expb15 have been found to be downstream of MIR164 and correlated with smaller seeds overall (Zheng et al. 2019). Furthermore, ZmEXPB15 has been found to underlie a maize kernel QTL, activated by ZmNAC11 and ZmNA (29, resulting in larger kernels through regulating nucellus elimination (Sun et al. 2022). In the 2019 study by Zheng et al., the promoter of expb14 was analyzed and found to be active in the endosperm, which was not observed in this study. This may be a result of differences in maize genotypes as the 2019 study was conducted using maize inbred line Mo17 and not LH244. Additionally, Zheng et al. examined an earlier phase of seed development, 9 DAP, which means this expression might have been missed by our analysis. Not mentioned in any of these studies is the activity of expb14 in developing vascular tissue, where it might be acting to activate expansion as well. Discoveries such as this may justify the additional work of checking many tissues for expression analyses.

### Root Specific Expression of Nas2

Nas2 is a nicotianamine synthase gene which creates nicotianamine (NA) from three molecules of S-adenosyl-L-methionine (Higuchi et al. 1999). NA itself is a precursor of phytosiderophores, which are secreted by grass roots to bind with Fe3+ which is then taken up by plasma membrane transporters (Römheld and Marschner 1986). In addition, NA is a chelator of metals broadly and can bind Fe, Mn, Cu, and Zn directly (Reichman and Parker 2002; Takahashi et al. 2003; Rellán-Alvarez et al. 2008; Haydon et al. 2012). Nas2 is a class I nicotianamine synthase and has been detected to be upregulated under Fe deficient conditions and downregulated under conditions of excess (Mizuno et al. 2003; Zhou et al. 2013). Unsurprisingly, the gene has been found to underlie a QTL conferring iron deficiency tolerance (Xu et al. 2022). The above observations regarding Nas2 broadly correspond to our observations from tests of the nas2 promoter, with the greatest expression seen in cortex tissues, where synthesis of NA is expected. The lack of expression in the primary crown root but presence of expression in the secondary roots is also not surprising given that secondary roots and root hairs are the primary site of nutrient uptake. The activity of the nas2 gene within leaf vasculature has not yet been noted in the literature. However, NA is known to be involved in the process of long-distance transport of FE and might be the reason why expression can be found there (Curie et al. 2009).

### REFERENCES

Abendroth, L. J., R. W. Elmore, M. J. Boyer, and S. K. Marlay. 2011. Corn growth and development. PMR 1009. Iowa State University Extension, Ames, Iowa.
Bansal, K C and Bogorad, L (1993) Cell type-preferred expression of maize cab-m1-repression in bundle sheath cells and enhancement in mesophyll cells. 90 (9): 4057-4061
Bae J M, Kwak M S, Noh S A, et al (2014) Overexpression of sweetpotato expansin cDNA (IbEXP1) increases seed yield in Arabidopsis. Transgenic Res 23:657-667. https://doi.org/10.1007/S11248-014-9804-1/FIGURES/5
Barkan, A and Walker, M (1994) Isolation and genomic mapping of cDNAs encoding the chloroplast Rieske Fe/S protein: two unlinked genes are transcribed in maize. 68:41
Broothaerts W, Mitchell H J, Weir B, et al (2005) Gene transfer to plants by diverse species of bacteria. Nature 433:629-633. https://doi.org/10.1038/nature03309
Castillo F M, Canales J, Claude A, Calderini DF (2018) Expansin genes expression in growing ovaries and grains of sunflower are tissue-specific and associate with final grain weight. BMC Plant Biol 18:1-14. https://doi.org/10.1186/S12870-018-1535-7/TABLES/3
Chen J C F, Tsai C C Y, Tzen J T C (1999) Cloning and Secondary Structure Analysis of Caleosin, a Unique Calcium-Binding Protein in Oil Bodies of Plant Seeds. Plant Cell Physiol 40:1079-1086. https://doi.org/10.1093/OXFORDJOURNALS.PCP.A029490
Christensen A H, Sharrock R A, Quail P H: Maize polyubiquitingenes: structure, thermal perturbation of expression and transcript splicing, and promoter activity following transfer to protoplasts by electroporation. Plant Mol Biol 18:675-689 (1992)
Curie C, Cassin G, Couch D, et al (2009) Metal movement within the plant: contribution of nicotianamine and yellow stripe 1-like transporters. Ann Bot 103:1-11. https://doi.org/10.1093/AOB/MCN207
David Freedman, Robert Pisani R P (2007) Statistics, 4th edn. W.W. Northon and Company, Inc, New York, New York
Dietz-Pfeilstetter A, Arndt N, Manske U (2016) Effects of a petunia scaffold/matrix attachment region on copy number dependency and stability of transgene expression in Nicotiana tabacum. Transgenic Res 25:149-162. https://doi.org/10.1007/S11248-015-9924-2/FIGURES/4
Engler C, Youles M, Gruetzner R, et al (2014) A Golden Gate modular cloning toolbox for plants. ACS Synth Biol 3:839-843. Erenstein O, Jaleta M, Sonder K, et al (2022) Global maize production, consumption and trade: trends and R&D implications. Food Secur 2022 145 14:1295-1319. https://doi.org/10.1007/S12571-022-01288-7
Faleiros R R S, Seebauer J R, Below F E (1996) Nutritionally Induced Changes in Endosperm of shrunken-1 and brittle-2 Maize Kernels Grown In Vitro. Crop Sci 36:947-954. https://doi.org/10.2135/CROPSCI1996.0011183X003600040022X
Gupta P K, Kulwal P L, Jaiswal V (2019) Association mapping in plants in the post-GWAS genomics era. Adv Genet 104:75-154. https://doi.org/10.1016/BS.ADGEN.2018.12.001
Hackett R (1993) GUS protocols: Using the GUS gene as a reporter of gene expression
Hanano A, Blée E, Murphy D J (2023) Caleosin/peroxygenases: multifunctional proteins in plants. Ann Bot 131:387-409. https://doi.org/10.1093/AOB/MCAD001
Han Y, Hu M, Ma X, et al (2022) Exploring key developmental phases and phase-specific genes across the entirety of anther development in maize. J Integr Plant Biol 64:1394-1410. https://doi.org/10.1111/JIPB.13276/SUPPINFO
Haydon M J, Kawachi M, Wirtz M, et al (2012) Vacuolar Nicotianamine Has Critical and Distinct Roles under Iron Deficiency and for Zinc Sequestration in Arabidopsis. Plant Cell 24:724-737. https://doi.org/10.1105/TPC.111.095042
Hernandez-Pinzon I, Patel K, Murphy D J (2001) The Brassica napus calcium-binding protein, caleosin, has distinct endoplasmic reticulum- and lipid body-associated isoforms. Plant Physiol Biochem 39:615-622. https://doi.org/10.1016/S0981-9428 (01) 01274-8
Higuchi K, Suzuki K, Nakanishi H, et al (1999) Cloning of Nicotianamine Synthase Genes, Novel Genes Involved in the Biosynthesis of Phytosiderophores. Plant Physiol 119:471-480. https://doi.org/10.1104/PP.119.2.471
Hily J-M, Singer S D, Yang Y, Liu Z (2009) A transformation booster sequence (TBS) from Petunia hybrida functions as an enhancer-blocking insulator in Arabidopsis thaliana. Plant Cell Rep 28:1095-104. https://doi.org/10.1007/s00299-009-0700-8
Hufford M B, Seetharam A S, Woodhouse M R, et al (2021) De novo assembly, annotation, and comparative analysis of 26 diverse maize genomes. Science 373:655-662. https://doi.org/10.1126/SCIENCE.ABG5289
Jiao Y, Peluso P, Shi J, et al (2017) Improved maize reference genome with single-molecule technologies. Nat 2017 5467659 546:524-527. https://doi.org/10.1038/nature22971
Kang M, Lee K, Finley T, et al (2022) An Improved Agrobacterium-Mediated Transformation and Genome-Editing Method for Maize Inbred B104 Using a Ternary Vector System and Immature Embryos. Front Plant Sci 13:860971. https://doi.org/10.3389/FPLS.2022.860971/BIBTEX
Klusch N, Dreimann M, Senkler J, et al (2022) Cryo-EM structure of the respiratory I+III2 supercomplex from Arabidopsis thaliana at 2 Å resolution. Nat Plants 2022 91 9:142-156. https://doi.org/10.1038/S41477-022-01308-6
Kumar K, Gambhir G, Dass A, et al (2020) Genetically modified crops: current status and future prospects. Planta 2020 2514 251:1-27. https://doi.org/10.1007/S00425-020-03372-8
Lazo G R, Stein P A, Ludwig R A (1991) A DNA Transformation-Competent Arabidopsis Genomic Library in Agrobacterium. Bio/Technology 1991 910 9:963-967. https://doi.org/10.1038/NBT1091-963
Li H, Yang M, Zhao C, et al (2021) Physiological and proteomic analyses revealed the response mechanisms of two different drought-resistant maize varieties. BMC Plant Biol 21. https://doi.org/10.1186/S12870-021-03295-W
Lizong H, Jinjie G, Yongfeng Z, et al (2014) Molecular Characterization and Evolutionary Analysis of the Putative Caleosin Gene Family in Maize (Zea mays). Int J Agric Biol
Lori Tausta S, Li P, Si Y, et al (2014) Developmental dynamics of Kranz cell transcriptional specificity in maize leaf reveals early onset of C4-related processes. J Exp Bot 65:3543-3555. https://doi.org/10.1093/JXB/ERU152
Lowe K, Wu E, Wang N, et al (2016) Morphogenic Regulators Baby boom and Wuschel Improve Monocot Transformation. Plant Cell 28:1998-2015. https://doi.org/10.1105/tpc.16.00124
Lu Z, Ricci W A, Schmitz R J, Zhang X (2018) Identification of cis-regulatory elements by chromatin structure. Curr Opin Plant Biol 42:90-94. https://doi.org/10.1016/j.pbi.2018.04.004
Mahmud K, Makaju S, Ibrahim R, Missaoui A (2020) Current Progress in Nitrogen Fixing Plants and Microbiome Research. Plants 2020, Vol 9, Page 97 9:97. https://doi.org/10.3390/PLANTS9010097
Mazaheri M, Heckwolf M, Vaillancourt B, et al (2019) Genome-wide association analysis of stalk biomass and anatomical traits in maize. BMC Plant Biol 19:1-17.
Mizuno D, Higuchi K, Sakamoto T, et al (2003) Three Nicotianamine Synthase Genes Isolated from Maize Are Differentially Regulated by Iron Nutritional Status. Plant Physiol 132:1989-1997. https://doi.org/10.1104/PP.102.019869
Mu X H, Chen Q W, Chen F J, Yuan L X, Mi G H (2017) A RNA-Seq analysis of the response of photosynthetic system to low nitrogen supply in maize leaf. 18 (12): 2624
Næsted H, Frandsen G I, Jauh G Y, et al (2000) Caleosins: Ca2+-binding proteins associated with lipid bodies. Plant Mol Biol 44:463-476. https://doi.org/10.1023/A: 1026564411918
Nguyen K Le, Grondin A, Courtois B, Gantet P (2019) Next-Generation Sequencing Accelerates Crop Gene Discovery. Trends Plant Sci 24:263-274. https://doi.org/10.1016/J.TPLANTS.2018.11.008
Nuccio M L (2018) A Brief History of Promoter Development for Use in Transgenic Maize Applications. Methods Mol Biol 1676:61-93. https://doi.org/10.1007/978-1-4939-7315-6_4
Patro R, Duggal G, Love M I, et al (2017) Salmon provides fast and bias-aware quantification of transcript expression. Nat Methods 2017 144 14:417-419. https://doi.org/10.1038/NMETH.4197
Pérez-González A, Caro E (2019) Benefits of using genomic insulators flanking transgenes to increase expression and avoid positional effects. Sci Reports 2019 91 9:1-11. https://doi.org/10.1038/S41598-019-44836-6
Pick T R, Bräutigam A, Schlüter U, et al (2011) Systems Analysis of a Maize Leaf Developmental Gradient Redefines the Current C4 Model and Provides Candidates for Regulation. Plant Cell 23:4208-4220. https://doi.org/10.1105/TPC.111.090324
Pörtner H O, Roberts D C, Tignor M, et al (2022) IPCC, 2022: Climate Change 2022: Impacts, Adaptation and Vulnerability. Cambridge, UK and New York, NY, USA
Raji J A, Frame B, Little D, et al (2018) Agrobacterium and Biolistic-Mediated Transformation of Maize B104 Inbred
Rajeevkumar S, Anunanthini P, Ramalingam S (2015) Epigenetic silencing in transgenic plants. Front Plant Sci 6:693. https://doi.org/10.3389/FPLS.2015.00693
Reichman S M, Parker D R (2002) Revisiting the Metal-Binding Chemistry of Nicotianamine and 2′-Deoxymugineic Acid. Implications for Iron Nutrition in Strategy II Plants. Plant Physiol 129:1435-1438. https://doi.org/10.1104/PP.005009
Rellán-Álvarez R, Abadía J, Álvarez-Fernández A (2008) Formation of metal-nicotianamine complexes as affected by pH, ligand exchange with citrate and metal exchange. A study by electrospray ionization time-of-flight mass spectrometry. Rapid Commun Mass Spectrom 22:1553-1562. https://doi.org/10.1002/RCM.3523
Ricci W A, Lu Z, Ji L, et al (2019) Widespread long-range cis-regulatory elements in the maize genome. Nat Plants 5:1237-1249.
Rocca N La, Barbato R, Vecchia F dalla, Rascio N (2000) Cab gene expression in bleached leaves of carotenoid-deficient maize. Photosynth Res 64:119-126. https://doi.org/10.1023/A: 1006477215572/METRICS
Römheld V, Marschner H (1986) Evidence for a Specific Uptake System for Iron Phytosiderophores in Roots of Grasses. Plant Physiol 80:175-180. https://doi.org/10.1104/PP.80.1.175
Sekhon R S, Briskine R, Hirsch C N, et al (2013) Maize Gene Atlas Developed by RNA Sequencing and Comparative Evaluation of Transcriptomes Based on RNA Sequencing and Microarrays. PLOS One 8. https://doi.org/10.1371/journal.pone.0061005
Simkin A J, McAusland L, Lawson T, Raines C A (2017) Overexpression of the RieskeFeS Protein Increases Electron Transport Rates and Biomass Yield. Plant Physiol 175:134-145. https://doi.org/10.1104/PP.17.00622
Singer S D, Hily J M, Cox K D (2011) Analysis of the enhancer-blocking function of the TBS element from Petunia hybrida in transgenic Arabidopsis thaliana and Nicotiana tabacum. Plant Cell Rep 30:2013-2025. https://doi.org/10.1007/S00299-011-1109-8/FIGURES/5
Stavolone L, Kononova M, Pauli S, et al (2003) Cestrum yellow leaf curling virus (CmYLCV) promoter: A new strong constitutive promoter for heterologous gene expression in a wide variety of crops. Plant Mol Biol 53:663-673.
Stelpflug S C, Sekhon R S, Vaillancourt B, et al (2015) An Expanded Maize Gene Expression Atlas based on RNA Sequencing and its Use to Explore Root Development. Plant Genome 9:0.
Sun Q, Li Y, Gong D, et al (2022) A NAC-EXPANSIN module enhances maize kernel size by controlling nucellus elimination. Nat Commun 2022 131 13:1-14. https://doi.org/10.1038/S41467-022-33513-4
Takahashi M, Terada Y, Nakai I, et al (2003) Role of Nicotianamine in the Intracellular Delivery of Metals and Plant Reproductive Development. Plant Cell 15:1263-1280. https://doi.org/10.1105/TPC.15.010256
Thompson C J, Movva N R, Tizard R, et al (1987) Characterization of the herbicide-resistance gene bar from Streptomyces hygroscopicus. EMBO J 6:2519-2523. https://doi.org/10.1002/j.1460-2075.1987.tb02538.x
Tibbs Cortes L, Zhang Z, Yu J (2021) Status and prospects of genome-wide association studies in plants. Plant Genome 14: e20077. https://doi.org/10.1002/TPG2.20077
Tnani H, López I, Jouenne T, Vicient C M (2011) Protein composition analysis of oil bodies from maize embryos during germination. J Plant Physiol 168:510-513. https://doi.org/10.1016/J.JPLPH.2010.08.020
Wang J, Jiang J, Oard J H (2000a) Structure, expression and promoter activity of two polyubiquitin genes from rice (Oryza sativa L.). Plant Sci 156:201-211. https://doi.org/10.1016/S0168-9452 (00) 00255-7
Wang J, Jiang J, Oard J H (2000b) Structure, expression and promoter activity of two polyubiquitin genes from rice (Oryza sativa L.). Plant Sci 156:201-211. https://doi.org/10.1016/S0168-9452 (00) 00255-7
Watson N R, Peschke V M, Russell D A, Sachs M M (1992) Analysis of 1-alanine: 2-oxoglutarate aminotransferase isozymes in maize. Biochem Genet 30:371-383. https://doi.org/10.1007/BF00569328/METRICS

What is claimed:

1. A construct comprising a promoter operably linked to a transgene, wherein the promoter is the promoter of a maize gene selected from the group consisting of ris2, Ihcb10, nas2, aa2m, and expb14 and portions or fragments thereof.

2. The construct of claim 1, wherein the construct is a vector.

3. The construct of claim 2, wherein the vector is an Agrobacterium vector.

4. The construct of claim 1, wherein the construct is a DNA donor template for CRISPR-mediated homology-directed repair.

5. The construct of claim 1, wherein the promoter comprises:
a) the native 2 kilobase sequence immediately upstream of the translation initiation site of the maize gene;
b) any one of SEQ ID NOs: 2-8; or
c) a functional fragment of (a) or (b).

6. The construct of claim 1, wherein the transgene encodes a reporter, a pharmaceutical, or a nutrient.

7. The construct of claim 1, wherein the transgene encodes a protein that
a) confers resistance to an infection, an insect, an herbicide, draught, waterlogging, or high temperatures; or
b) confers an increase in maize yield or nutritional content.

8. The construct of claim 1, wherein the construct allows for tissue-specific expression and is selected from the group consisting of ris2, Ihcb10, and fragments thereof.

9. A method for driving expression of a transgene in a maize plant, the method comprising: introducing the construct of claim 1 into the maize plant.

10. The method of claim 9, wherein the construct is introduced via agrobacterium-mediated transformation.

11. The method of claim 9, wherein the construct is introduced via CRISPR-mediated homology-directed repair.

12. The method of claim 9, wherein the construct is incorporated into the genome of the maize plant.

13. The method of claim 9, wherein the construct is maintained as extrachromosomal DNA.

14. The method of claim 9, wherein the expression is embryo-specific, root-specific, anther-specific, or leaf-specific.

15. The method of claim 14, wherein the expression is embryo-specific and the promoter is the aa2m or expb14 promoter or a fragment thereof.

16. The method of claim 14, wherein the expression is root-specific and the promoter is the nas2 promoter or a fragment thereof.

17. The method of claim 14, wherein the expression is anther-specific and the promoter is the lhcb10 or ris2 promoter or a fragment thereof.

18. The method of claim 14, wherein the expression is leaf-specific and the promoter is the lhcb10 or ris2 promoter or a fragment thereof.

19. A transgenic maize plant comprising the construct of claim 1.

20. The transgenic maize, wherein the plant was produced via the method of claim 9.
